(19) United States
(12) Patent Application Publication
(10) Pub. No.: US 2017/0051305 A1
(43) Pub. Date: Feb. 23, 2017

(54) HYBRID BREEDING METHOD FOR FACULTATIVE APOMICTIC PLANTS

(71) Applicant: YULEX CORPORATION, Chandler, AZ (US)

(72) Inventors: Robert Schmidt, Rancho Santa Fe, CA (US); Eric Mathur, Encinitas, CA (US)

(21) Appl. No.: 15/308,337

(22) PCT Filed: May 5, 2015
(86) PCT No.: PCT/IB15/01627

§ 371 Date: Nov. 1, 2016

Related U.S. Application Data
(60) Provisional application No. 61/989,065, filed on May 6, 2014.

(51) Int. Cl. C12N 15/82; A01H 1/02

(52) U.S. Cl. CPC C12N 15/8287; A01H 1/02

(57) ABSTRACT

The present disclosure relates to materials and methods useful for improving the efficacy of a plant breeding program such as, for example, the method for producing hybrid seeds in a facultative apomictic crop species, which in turns are useful for, for example, commercial production of highly uniform hybrid progeny. Hybrid seeds produced by such improved breeding methods, and plant grown from such hybrid seeds are also within the scope of the present invention. The disclosure further relates to processes for making a plant-derived product derived from any of the foregoing hybrid plants, and plant-derived products produced by such processes.

### CROSS-REFERENCE TO RELATED APPLICATIONS

This application claims the benefit of priority of U.S. Provisional Application Ser. No. 61/989,065, filed May 6, 2014, the contents of which are herein incorporated by reference in their entirety.

### FIELD OF THE INVENTION

The present disclosure generally relates to the field of agriculture, in particular to new hybrid plants and processes for obtaining them. More specifically, the disclosure relates to methods for improving the efficacy of a plant breeding program such as, for example, the method for producing hybrid seeds in a facultative apomictic crop species that will breed true for highly uniform progeny. The disclosure further relates to hybrid seeds produced by such improved breeding methods, to hybrid plants with improved agronomic characteristics, and to bio-products derived from such hybrid plants.

### BACKGROUND OF THE INVENTION

Reproduction in plants is ordinarily classified as sexual or asexual. The term apomixis is generally accepted as the replacement of sexual reproduction by various forms of asexual reproduction. Mechanistically, apomixis is a genetically controlled method of reproduction in plants where the embryo is formed without union of an egg and a sperm. In many cases, an embryo can be formed apomictically from a chromosomally unreduced megaspore mother cell or from a somatic cell of the nucellus or ovule. Apomixis has economic potential because it can cause any genotype, regardless of how heterozygous, to breed true, in part because it is a reproductive process that bypasses female meiosis and syngamy to produce embryos genetically identical to the maternal parent. As a result, apomixis makes vegetative reproduction or cloning through the seed possible. With apomictic reproduction, progeny of especially adaptive or hybrid genotypes would maintain their genetic fidelity throughout repeated life cycles, which in turn can potentially provide a way to fix vigor by allowing a plant to clone itself indefinitely through seed. In addition to fixing hybrid vigor, apomixis can make possible commercial hybrid production in crops where efficient male sterility or fertility restoration systems for producing hybrids are not known or developed. Further, apomixis could have a major impact in commercial hybrid production systems by simplifying hybrid seed production and therefore making hybrid development more efficient.

Guayule (Parthenium argentatum Gray), which is a member of the Asteraceae family, has been long-recognized as a promising alternative source of natural rubber with the potential for cultivation in the arid and semi-arid environments. In fact, among >2,000 rubber-producing species, guayule is the only species, besides Hevea brasiliensis (rubber tree), that has been utilized as a source of natural rubber on a commercial scale. Guayule, with its higher concentration of resin and lower concentration of protein, is generally considered a superior and more efficient adhesive plant. This conclusion is based on the physical and chemical structure of both the resin and rubber. In the early part of the last century, guayule rubber was commercially-produced, however its production have not significantly expanded because the production costs of bulk rubber for tire manufacture were too high to permit direct competition with Hevea rubber. In recent years, guayule commercialization has been revitalized by use of the plant to produce low protein latex, which causes mild to severe reactions in Type I Hevea latex allergic people. This application allows a higher value rubber raw material and commercial competitiveness. However, while Hevea is a somewhat established and improved crop, acclimated to growth in areas outside of its natural habitat, work is still underway to completely domesticate and commercialize guayule as an alternative crop for arid and semiarid areas.

The global market for industrial rubber products is projected to increase 5.8 percent per year to $140 billion in 2016, according to a report published by The Freedonia Group. The demand for rubber has been driven by high growth rates in developing economies, and the resurgence in major end-use markets. Strong surge in manufacturing activity across various industries, ranging from medical devices, household appliances to construction machinery and automotive vehicles is expected to foster demand for industrial rubber products, particularly in developing markets. Further, expanding application areas and increasing ease of processing are also expected to favor strong growth of rubber market. However, several countries, including the United States and European countries, are almost entirely dependent on imports of natural rubber for industrial purposes. In fact, the annual cost of importing natural rubber from tropical and subtropical rubber production hubs amounts to nearly $2.5 billion in the United States alone.

Therefore, to help meet the growing needs for natural rubber, the guayule industry has to keep growing to meet the needs of the consuming public. Successful commercialization of guayule depends largely upon the development of higher yielding cultivars from the available germplasm. A number of guayule breeding programs have been initiated to improve its rubber yield and quality. The primary objective of these guayule breeding programs has been to facilitate successful commercialization by developing higher yielding cultivars. These breeding efforts have been aimed primarily at increasing the rubber content and overall yield with the most recent efforts focused on single-plant selections among polyploidy apomictic plants and interspecific hybridization with other Parthenium species (Thompson and Ray, 1989). However, breeding for increased rubber yield has been difficult because the most commonly used varieties of guayule are polyploid and reproduce apomictically at variable percentages.

As a result, substantial research and development efforts are devoted to the modernization of planting and harvesting of fields and processing of guayule, and to the development of economically advantageous guayule varieties. However, when breeding an undomesticated crop such as guayule, plant breeders are often working with an unfamiliar species that is not yet fully domesticated and the available germplasm is often limited. In addition, although guayule reproduces predominantly by apomixis, it is in fact a facultative apomictic species in which apomixis and sexuality coexist. Its facultative nature and the high amount of heterozygosity in individual plants and the heterogeneous make-up of populations, results in the release of considerable variation whenever sexual reproduction, i.e. amphimixis, occurs and periodically releases genetic variation among progeny. For example, the origin and relative chromosome numbers of four different classes of progeny from tetraploid (2n=4x=72) parents (Ray et al., Industrial Crops Products, 22:15-25, 2005) illustrates the complexity of the reproductive biology in this plant species.

Nevertheless, in guayule there have been some successes through plant breeding, shown by dramatic increases in latex yield from 300 to 1000 kg/ha rubber for guayule. Success of such efforts is greatly enhanced by the identification of relevant genes of interest and recent development of guayule transgenesis. Improvements in growth and latex production, production of non-allergenic latex and plant products, together with environmental benefits will increase the returns from guayule plantations on eroded land, and encourage adoption by small and marginal farmers in the tropics. However, guayule still contains many wild characteristics that are deterrents to full commercialization. This is in part because all current breeding approaches depend upon the existing genetic variability found in the available germplasm. In guayule, this genetic base appears to be very narrow, even though the facultative nature of apomixis in polyploid guayule continually releases new variability with each seed harvest due to the re-shuffling of the pre-existing allele combinations. As guayule approaches commercialization, guayule breeding will inevitably become a priority and various breeding schemes should be tested and utilized.

Thus, there is a long-standing and continuing need for new methods for optimizing breeding strategies for producing highly uniform hybrid progeny with agronomically desirable genotypes. This and other needs are addressed by the presently disclosed subject matter. For example, the present disclosure provides new breeding methods for the commercial production of uniform apomictic hybrids in facultative apomictic plants such as, for example, guayule with the desirable characteristics such as, for example, good processing for industrial purposes, high latex and rubber yield, high resin content, resistance to diseases and pests, and greater adaptability to various growing areas and conditions.

### SUMMARY OF THE INVENTION

The following embodiments and aspects thereof are described in conjunction with systems, materials, and methods that are meant to be exemplary and illustrative, not limiting in scope. In various embodiments, one or more of the above-described needs have been reduced or eliminated, while other embodiments are directed to other improvements. Any embodiment discussed herein with respect to one aspect of the invention applies to other aspects of the invention as well, unless specifically noted.

In one aspect, the present invention provides a plant breeding method for the production of hybrid seed. The breeding method includes (a) pollinating an essentially self-incompatible diploid plant as female parent with pollen from a tetraploid male parent to produce one or more F1 triploid hybrid seeds on the female parent; (b) selecting an apomictic triploid hybrid plant grown from the one or more F1 triploid hybrid seeds; (c) clonally propagating the apomictic triploid hybrid plant to produce a cloned apomictic plant line; and (d) growing one or more plants of the cloned apomictic plant line, and collecting resulting apomictically-derived hybrid seeds from the grown plants.

In another aspect, the present invention provides a plant breeding method for the production of substantially hybrid seed, including: (a) pollinating a tetraploid plant as female parent with pollen from a hexaploid male parent to produce one or more F1 hybrid seeds on the female parent; (b) selecting an F1 apomictic hybrid plant grown from the one or more F1 hybrid seeds; (c) clonally propagating said F1 pentaploid hybrid plant to produce a cloned apomictic plant line; and (d) growing one or more plants of the apomictic plant line and collecting resulting apomictically-derived hybrid seeds from the grown plants. In a preferred embodiment of this aspect, the one or more F1 hybrid seeds of step (b) are further defined as pentaploid (5N) or heptaploid (7N) hybrid seeds. In another preferred embodiment, the one or more F1 hybrid seeds of step (b) are further defined as pentaploid hybrid seeds.

These and other embodiments of the breeding methods disclosed herein can optionally include one or more of the following features. In one embodiment, at least one of the two parental plants is pre-selected for high productivity prior to the pollination step (a). In another embodiment, the plants grown from the hybrid seeds of step (b) are further selected for high productivity prior to clonal propagation step (c). In another embodiment, the female parent or the male parent or both parents are essentially homozygous plants or plants of inbred lines. In yet another embodiment, the female parent and the male parent are genetically distinct. One significant advantage of producing hybrid progeny by genetically mating two genetically distinct and distant parents is to potentially maximize the hybrid vigor, or heterosis, among the resulting progeny.

In a further non-limiting embodiment of any of the foregoing methods, the clonal propagation step (c) is achieved by rooted cutting, stem cutting, stake cutting, tissue-culture, or any of other means of vegetative propagation. In another embodiment, the apomictically-derived hybrid seeds from step (d) are further planted to produce hybrid crop plants. In a preferred embodiment, the apomictically-derived hybrid seeds produced by any of the forgoing plant breeding methods can be grown to generate substantially uniform hybrid progeny. In another embodiment, the female and male parents according to any one of the foregoing are plants of a facultative apomictic species. In a preferred embodiment, the facultative apomictic species preferably belongs to a family selected from the group consisting of Asteraceae, Orchidaceae, Poaceae, and Rosaceae. In another preferred embodiment, the facultative apomictic species belongs to a genus selected from the group consisting of Agropyrum, Allium, Amelanchier, Antennaria, Beta, Boechera, Brachiaria, Cenchrus, Chloris, Compositae, Coprosma, Cortaderia, Crataegus, Cytrus, Datura, Dichanthium, Eragrostis, Erigeron, Eriochloa, Eupatorium, Heteropogon, Hieracium, Hyparrhenia, Hypericum, Ixeris, Panicum, Parthenium, Paspalum, Paspalum, Pennisetum, Poa, Ranunculus, Rubus, Sorghum, Taraxacum, Themeda, Tripsacum, and Urochloa. In yet another preferred embodiment, the facultative apomictic species is further defined as Parthenium argentatum.

Also provided, in another aspect of the present invention, is a hybrid seed produced by a plant breeding method according to any of the foregoing methods. In yet another aspect, the present invention further provides a hybrid plant grown from such seed. In a preferred embodiment of this aspect, the hybrid plant exhibits an improved target trait. In a preferred embodiment, the improved target of the hybrid plant is selected from the group consisting of high productivity, high latex yield, high resin yield, high overall rubber yield, abiotic stress tolerance, biotic stress tolerance, disease resistance, improved water use efficiency, improved nitrogen use efficiency, and combinations of any thereof. In a particularly preferred embodiment, the improved target trait of the hybrid plant is further defined as high latex productivity or high biomass. In a further embodiment of this aspect of the present invention, the hybrid plant further includes a transgene. In a preferred embodiment, the transgene confers a trait selected from the group consisting of high productivity, high latex yield, high resin yield, high overall rubber yield, abiotic stress tolerance, biotic stress tolerance, disease resistance, improved water use efficiency, improved nitrogen use efficiency, and combinations of any thereof.

In one aspect, the present invention further provides a seed, a reproductive tissue, a vegetative tissue, a plant part, a biomass, or progeny of a hybrid plant disclosed herein. In one embodiment of this aspect, provided herein is a plant part of a hybrid plant disclosed herein, wherein the plant part is selected from the group consisting of a cell, a protoplast, an inflorescence, a flower, a sepal, a petal, a pistil, a stigma, a style, an ovary, an ovule, an embryo, a seed, a stamen, a filament, an anther, a male gametophyte, a female gametophyte, a pollen grain, a meristem, a terminal bud, an axillary bud, a leaf, a stem, a root, a cell of said plant in culture, a tissue of said plant in culture, an organ, a cutting, an explant, and a callus.

Further provided, in one aspect of the present invention, is a method for producing a plant-derived product. The method according to this aspect of the invention includes obtaining a hybrid plant grown from a hybrid seed produced by any of the foregoing methods, or a part thereof, and producing said plant-derived product therefrom. As such, additionally provided, in another aspect of the present invention, is a plant product produced by a process of producing a plant-derived product disclosed herein. In some embodiments of this aspect, the plant derived-product is selected from the group consisting of latex, resin, fatty acid triglycerides, terpenes, sesquiterpenes, or waxes. In some preferred embodiments of this aspect, the plant derived-product is further defined as a latex product. In some particularly preferred embodiments, the latex product is selected from the group consisting of medical gloves, surgical gloves, elastic bands, elastic traps, condom, automobile tires, truck tires, airplane tires, and wet suits.

In addition to the exemplary aspects and embodiments described above, further aspects and embodiments will become apparent by study of the following descriptions. It should be understood, however, that the detailed description and any specific examples provided, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1. Tetraploid guayule plants display facultative apomixes.

FIG. 2. Values resulting from IBS analysis of SNP calls. The values along the diagonal (bolded) indicate the number of SNP sites with genotype calls passing the filters applied to the dataset for each individual. The values below the diagonal indicate the total number of IBS comparisons per pair of individuals (4×total common SNPs). The values above the diagonal indicate the number of these comparisons that were identical between the two individuals.

FIG. 3. Development of high-yielding and substantially uniform guayule hybrid lines.

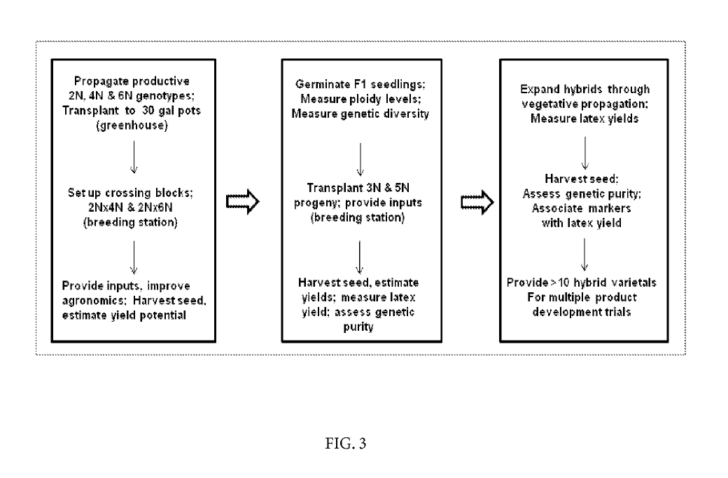

FIG. 4. Genotypes with triploid 3N or pentaploid 5N genomes are selected for the development of pre-commercial hybrids.

FIG. 5. Improvement of parental breeding lines through trait introgression.

### DETAILED DESCRIPTION OF THE INVENTION

The present disclosure relates to materials and methods useful for improving the efficacy of a plant breeding program such as, for example, the method for producing hybrid seeds in a facultative apomictic crop species, which in turns are useful for, for example, commercial production of highly uniform F1 hybrid progeny. Hence, hybrid seeds produced by such improved breeding methods, and plant grown from such hybrid seeds are also within the scope of the present invention. Further disclosed herein are processes for making a plant-derived product derived from any of the foregoing hybrid plants, and plant-derived products produced by such processes.

### SOME DEFINITIONS

Unless otherwise defined, all terms of art, notations and other technical and scientific terms or terminology used herein are intended to have the meanings commonly understood by those of skill in the art to which this invention pertains. Many of the techniques and procedures described or referenced herein are well understood and commonly employed using conventional methodology by those skilled in the art. The following terms are defined for purposes of the invention as described herein. In some cases, terms with commonly understood meanings are defined herein for clarity and/or for ready reference, and the inclusion of such definitions herein should not necessarily be construed to represent a substantial difference over what is generally understood in the art.

The singular forms “a”, “an”, and “the” include the plural reference unless the context clearly dictates otherwise. Thus, for example, a reference to “a host cell” includes a plurality of such host cells, and a reference to “a stress” is a reference to one or more stresses and equivalents thereof known to those skilled in the art, and so forth.

“Apomixis”, as used herein, in flowering plants is defined as the asexual formation of a seed from the maternal tissues of the ovule, avoiding the processes of meiosis and fertilization, leading to embryo development. All known mechanisms of apomixis share three developmental components: the generation of a cell capable of forming an embryo without prior meiosis (apomeiosis); the spontaneous, fertilization-independent development of the embryo (parthenogenesis); and the capacity to either produce endosperm autonomously or to use an endosperm derived from fertilization.

As used herein, “ASE” is the abbreviation for ‘accelerated solvent extraction’ and refers to an automated rubber extraction instrument which sequentially extracts rubber from small amounts of ground plant materials using hexane or other organic solvent. The ASE instrument is used to quantify rubber content in guayule cultivars.

“Biomass”. As used herein, plant biomass refers to the amount of (e.g., measured in grams of air-dry tissue) of a harvestable plant tissue produced from the plant in a growing season, which could also determine or affect the plant yield or the yield per growing area. Non-limiting examples of such harvestable plant tissues include leaves, stems, and reproductive structures, or all plant tissues such as leaves, stems, roots, and reproductive structures.

The term “crossing” as used herein refers to the fertilization of female plants (or gametes) by male plants (or gametes). The term “gamete” refers to the reproductive cell (egg or sperm) produced in plants by mitosis from a gametophyte and involved in sexual reproduction, during which two gametes of opposite sex fuse to form a zygote. The term generally includes reference to a pollen (including the sperm cell) and an ovule (including the ovum). “Crossing” therefore generally refers to the fertilization of ovules of one individual with pollen from another individual, whereas “selfing” refers to the fertilization of ovules of an individual with pollen from the same individual. Crossing is widely used in plant breeding and results in a mix of genomic information between the two plants crossed one set of chromosomes from the mother and one set of chromosomes from the father. This will result in a new combination of genetically inherited traits. Usually, the progeny of a crossing is designated as: “F1”. If the F1 is not uniform (segregates) it is usually designated as “F1 population”. “Selfing” of a homozygous plant will usually result in a genetic identical plant since there is no genetic variation. “Selfing” of an F1 will result in an offspring that segregates for all traits that have heterozygotic loci in the F1. Such offspring is designated: “F2” or “F2 population”.

The term “polycross” or “polycrossing” refers to a cross used in selective plant breeding which is a method of mass experimental crossbreeding. It involves finding clones of strains which, upon crossbreeding with other clones or strains of the same species, yield the most productive plants. The resulting plants are used in developing a new “synthetic variety.” This method is commonly used in the selective breeding of plants that can be successfully cloned such as guayule, as well as other perennial herbs and annuals and biennials that propagate vegetatively. The term “intercrossable”, as used herein, refers to the ability to yield progeny plants after making crosses between parent plants.

“Cross-pollination”. As used herein, the term “cross-pollination” refers to fertilization by the union of two gametes from different plants. A plant is cross-pollinated if the pollen comes from a flower on a different plant from a different family or line. Cross-pollination does not include sib- and self-pollination.

“Cultivar”. As used herein, a “cultivar” or a “variety” refers to a group of similar plants that belong to the same species and that, by structural features and performance, may be distinguished from other varieties within the same species. Two essential characteristics of a variety are identity and reproducibility. Identity is necessary so that the variety may be recognized and distinguished from other varieties within the crop species. The distinguishing features may be morphological characteristics, molecular markers, color markings, physiological functions, disease reaction, or performance. Most agricultural varieties are pure for the characteristic or for those characteristics that identify the variety; per se. Reproducibility is needed in order that the characteristic(s) by which the variety is identified will be reproduced in the progeny. For the purpose of this disclosure, therefore, the terms “cultivar” and “variety” are used interchangeably to refer to a group of plants within a species (here, Parthenium argentatum) that share certain constant characters which separate them from the typical form and from other possible varieties within that species. While possessing at least the distinctive trait, a “variety” of the invention also may be characterized by a substantial amount of overall variation between individuals within the variety, based primarily on the Mendelian segregation of traits among the progeny of succeeding generations. On the other hand, “cultivar” or “variety” also can denote a cloned line, since a Parthenium argentatum cultivar may individually be reproduced asexually, via stem cuttings, and all of the clones would be essentially identical genetically.

A “line”, as used herein, refers to a population of plants derived from a single cross, backcross or selfing. The individual offspring plants are not necessarily identical to one another. As distinguished from a “variety,” a “line” displays less variation between individuals, generally (although not exclusively) by virtue of several generations of self-pollination. For purposes of this disclosure, a “line” is defined sufficiently broadly to include a group of plants vegetatively or clonally propagated from a single parent plant, using stem cuttings or tissue culture techniques.

The term “breeding line”, as used herein, refers to a line of a cultivated crop having commercially valuable or agronomically desirable characteristics, as opposed to wild varieties or landraces. The term includes reference to an elite breeding line or elite line, which represents an essentially homozygous, usually inbred, line of plants used to produce commercial F1 hybrids. An elite breeding line is obtained by breeding and selection for superior agronomic performance comprising a multitude of agronomically desirable traits. An elite plant is any plant from an elite line. Elite breeding lines are essentially homozygous and are preferably inbred lines.

Genotype, as used herein, refers to the genetic constitution of a cell or organism.

Haploid and doubled-haploid: A haploid cell or organism having one set of the two sets of chromosomes in a diploid. Doubled haploids are plants that have two copies of each chromosome, (2n), like diploids. However, they differ from diploids in that they were created from a single grain of pollen, an ovum, or indeterminate gametes that are cultured. Their chromosomes doubled through chemical means, and the cultured tissue grown into a plant. The haploid genome of the gametes, when doubled, produced a plant with a complete genome, with two identical copies of every gene. Thus, double haploids are homozygous at every locus, and can have highly variable phenotypes. Double haploids have been made for many plant species to assist in breeding experiments.

As used herein, the term “homozygous” means a genetic condition existing when identical alleles reside at corresponding loci on homologous chromosomes. Homozygosity levels are average values for the population, and refer preferably to those loci at which the parental genomes are identical. The expression “essentially homozygous line” refers to a plant line having a level of homozygosity of at least 90%, preferably at least 95%, preferably at least 96%, more preferably at least 97%, more preferably still at least 98%, and most preferably at least 99% or at least 100% homozygosity when testing at least 50, preferably at least 100, preferably at least 300, more preferably at least 500, more preferably at least 1,000; more preferably at least 2,000; and most preferably at least 10,000 loci. In some preferred embodiments, the homozygosity level is determined using molecular methods.

As used herein, the term “heterozygous” means a genetic condition existing when different alleles reside at corresponding loci on homologous chromosomes. The expression “heterozygous line” merely reflects that the line is not an “essentially homozygous line” as used herein.

As used herein, the term “hybrid” means any offspring of a cross between two genetically non-identical individuals. The parental plants may be related, as in production of a modified single cross, or unrelated. F₁ hybrid, as used herein, refers to the first generation progeny of the cross of two genetically dissimilar plants.

As used herein, the terms “introgressing”, “introgress” and “introgressed” refer to both a natural and artificial process whereby individual genes or entire chromosomes are moved from one individual, species, variety or cultivar into the genome of another individual, species, variety or cultivar, by crossing those individuals, species, varieties or cultivars. In plant breeding, the process usually involves selfing or backcrossing to the recurrent parent to provide for an increasingly homozygous plant having essentially the characteristics of the recurrent parent in addition to the introgressed gene or trait.

The expressions “latex content”, “rubber content”, and “resin content”, as used herein, refer to the amount of latex, rubber, and resin respectively, in a given plant organ or tissue, such as the stem (seed oil content) and is typically expressed as percentage of dry weight (for example at 10% humidity of biomass) or wet weight. It should be noted that latex, rubber, and resin content is affected by intrinsic latex, rubber, and resin production of a tissue (e.g., stem, leaf), as well as the mass or size of the latex-producing tissue per plant or per growth period. In one embodiment, increase in latex, rubber, or resin content of the plant can be achieved by increasing the size/mass of a plant's tissue(s) which contains latex, rubber, and resin per growth period. Thus, increased latex, rubber, and/or resin content of a plant can be achieved by increasing the yield, growth rate, biomass and vigor of the plant.

A “locus” is defined herein as the position that a given gene occupies on a chromosome of a given plant species. A locus confers one or more traits such as, for example, male sterility, female-only flower, herbicide tolerance, pest resistance, disease resistance, synchronous germination, synchronous flowering, early flowering, improved plant yield and/or fruit yield, modified plant architecture, abiotic stress tolerance, modified fatty acid metabolism, modified oil content, modified carbohydrate metabolism, and modified protein metabolism. The trait may be, for example, conferred by a naturally occurring gene introduced into the genome of the variety by backcrossing, a natural or induced mutation, or a transgene introduced through genetic transformation techniques. A locus may comprise one or more alleles integrated at a single chromosomal location.

Phenotype is defined herein as the detectable characteristics of a cell or organism, which characteristics are the manifestation of gene expression.

The term “plant” refers to any living organism belonging to the kingdom Plantae. As used herein, the term “plant” includes reference to an immature or mature whole plant, including a plant from which seed or anther have been removed. For the purpose of this disclosure, a seed or embryo that will produce the plant is also considered to be the plant.

Plant characteristic: A plant characteristic can be a morphological, physiological, agronomic, or genetic feature of a plant.

Plant growth. This term refers to the process by which plants increase in size and mass. The increase in the number and size of plant organs is directly associated with an increase in cell numbers and/or cell size, which involves cell division, growth, expansion and differentiation. Plant growth can be generally divided into vegetative and reproductive growth in the life cycle.

The term “plant part” refers to any part of a plant including, but not limited to, organelles, single cells and cell tissues such as plant cells that are intact in plants, cell clumps and tissue cultures from which guayule plants can be regenerated. Examples of plant parts include, but are not limited to, single cells and tissues from pollen, ovules, leaves, embryos, roots, root tips, tubers, anthers, flowers, fruits, stems shoots, and seeds; as well as pollen, ovules, leaves, embryos, roots, root tips, anthers, flowers, fruits, stems, shoots, scions, rootstocks, seeds, tubers, protoplasts, calli, and the like. The two main parts of plants grown in some sort of media, such as soil, are often referred to as the “above-ground” part, also often referred to as the “shoots”, and the “below-ground” part, also often referred to as the “roots”.

As used herein, the term “progeny” means (a) genetic descendant(s) or offspring. “Progeny” includes descendants of a particular plant or plant line. Progeny of a plant according to the present invention include seeds formed on F₁, F₂, F₃, F₄, F₅, F6 and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F2, F3, F₄, F₅ and F₆ refer to subsequent generations of self- or sib-pollinated progeny of an F1 plant.

Resistance or tolerance: As used herein, the terms “resistance” and “tolerance” are used interchangeably to describe a plant having the ability to prevent, decrease, or repair the injury induced by a specified biotic or abiotic stress on a plant or a plant population such as insect pest, pathogenic disease, abiotic influence, or environmental condition. These terms are also used to describe plants showing some stress symptoms but that are still able to produce marketable product with an acceptable yield. Some plants that are referred to as resistant or tolerant are only so in the sense that they may still produce a crop, even though the plants are stunted and the yield is reduced.

The term “regeneration” as used herein refers to the development of a plant from tissue culture.

The term “RSD” as used herein refers to the standard deviation (RSD or % RSD) and is the absolute value of the coefficient of variation. It is often expressed as a percentage.

“Selfing” refers to the manifestation of the process of “self-pollination”, which in turn refers to the transfer of pollen from the anther of a flower to the stigma of the same flower or different flowers on the same plant. The term “selfing” therefore refers to the process of self-fertilization wherein an individual is pollinated or fertilized with its own pollen. Repeated selfing eventually results in homozygous offspring.

As used herein, the term “SNP” is the abbreviation for “single nucleotide polymorphism” which is a DNA sequence variation occurring commonly within a population in which a single nucleotide—A, T, C or G—in the genome differs between members of a biological species or paired chromosomes.

As used herein, the term “tissue culture” refers to a composition comprising isolated plant cells of the same or a different type or a collection of such cells organized into parts of a plant, in which the cells are propagated in a nutrient medium under controlled conditions. Non-limiting examples of tissue cultures include plant protoplasts, plant cell tissue culture, culture microspores, plant calli, plant clumps, and the like. As use herein, phrases such as “grown the seed” or “grown from the seed” include embryo rescue, isolation of cells from seed for use in tissue culture, as well as traditional growing methods.

“Vegetative propagation”, as used herein, refers to asexual propagation of the plant that is accomplished by taking and propagating cuttings, by grafting or budding, by layering, by division of plants, or by separation of specialized structure, such as stem, roots, tubers, rhizomes, or bulbs.

As used herein the phrase “plant vigor” refers to the amount (measured by weight) of tissue produced by the plant in a given time. Hence increased vigor could determine or affect the plant yield or the yield per growing time or growing area. In addition, early vigor (seed and/or seedling) often results in improved field stand establishment. As used herein, stand establishment refers to the survivability and density of areas of land newly planted with guayule, typically by seed or stem propagation.

As used herein the phrase “plant yield” refers to the amount (as determined by, e.g. volume, weight or size) or quantity (numbers) of tissues or organs or plant-derived materials, such as latex or resin, produced per plant or per growing season. Hence increased yield could affect the economic benefit one can obtain from the plant in a certain growing area and/or growing time. It should be noted that a plant yield can be affected by various parameters including, but not limited to, plant biomass; plant vigor; growth rate; latex yield; latex quantity; latex quality in harvested organs (e.g., reproductive or vegetative parts of the plant); harvest index; number of plants grown per area; number and size of harvested organs per plant and per area; number of plants per growing area (density); number of harvested organs in field; total leaf area; carbon assimilation and carbon partitioning (the distribution/allocation of carbon within the plant); resistance to shade; number of harvestable organs (e.g. stem or leaf), weight per plant; and modified plant architecture.

As used herein the phrase “latex yield” refers to the amount, volume, or weight of crude latex per plant, per growing season, or per growing area. Hence latex yield can be affected by plant dimensions (e.g., length, width, perimeter, area and/or volume), or by number of plants per growing area. Hence an increase of latex yield per plant could affect the economic benefit one can obtain from the plant in a certain growing area and/or growing time; and an increase of latex yield per growing area could be achieved by increasing latex yield per plant, and/or by increasing number of plants grown on the same given area.

### Apomixis and Facultative Apomicts

The term apomixis is generally accepted as the replacement of sexual reproduction by various forms of asexual reproduction. Mechanistically, apomixis is a genetically controlled method of reproduction in plants where the embryo is formed without union of an egg and a sperm. Apomixis affects both megasporegenesis and megagametogenesis, by typically does not alter pollen formation. Meiosis still occurs normally in the anthers, and viable, reduced pollen is usually produced in both aposporous and diplosporous apomicts. There are three basic types of apomictic reproduction: 1) apospory—embryo develops from a chromosomally unreduced egg in an embryo sac derived from the nucellus, 2) diplospory—embryo develops from an unreduced egg in an embryo sac derived from the megaspore mother cell, and 3) adventitious embryony—embryo develops directly from a somatic cell. In most forms of apomixis, pseudogamy or fertilization of the polar nuclei to produce endosperm is necessary for seed viability.

Introducing apomixis in a breeding program could have several advantages. Apomixis has important economic potential because they can cause any genotype, regardless of how heterozygous, to breed true. Since it is a reproductive process that bypasses female meiosis and syngamy to produce embryos genetically identical to the maternal parent, progeny of highly adaptive or hybrid genotypes with apomictic reproduction would maintain their genetic fidelity throughout repeated life cycles. Therefore, the genotype of every apomictic would be fixed in the F1 generation and every apomictic genotype from a cross has the potential of being a cultivar. Gene combinations and vigor would not be lost as in each segregating generation of sexual F1 hybrids. The maintenance of elite genotypes would be therefore easier and more efficient. In addition to fixing hybrid vigor, apomixis can make possible commercial hybrid production in crops where efficient male sterility or fertility restoration systems for producing hybrids are not known or developed. Further, apomixis could have a major impact in commercial hybrid production systems by simplifying hybrid seed production and therefore making hybrid development more efficient.

Apomixis is said to be facultative when some progeny also result from either a normal meiosis and/or a normal fertilization of the egg cell. Apomixis is said to be obligate when the progeny is 100% maternal.

Guayule (Parthenium argentatum Gray)

Guayule (Parthenium argentatum Gray), which is a member of the Asteraceae family, has been considered a potential economic and renewable source of rubber. Besides Hevea brasiliensis (rubber tree), guayule has been increasingly utilized as a source of natural rubber on a commercial scale. Today, Hevea is an established and greatly improved crop, acclimated to growth in areas outside of its natural habitat. In contrast, work is still underway to completely domesticate and commercialize guayule as a new or alternative crop for arid and semiarid areas. A number of guayule breeding programs are also under way to improve its rubber yield and quality.

Successful commercialization of guayule also depends on utilizing as much of the plant as possible. Initially, latex is the primary product, but the residual plant material or bagasse also contributes to guayule's future economic development. Natural rubber is a biopolymer of cis-1,4-polyisoprene with 400-50,000 isoprene units enzymatically linked in a head-to-tail configuration. It is formed by a branch of the isoprenoid pathway which also leads to the production of dimers, trimers, tetramers, and so forth. These lower molecular weight molecules and various isomers constitute the resin. In common practices, only approximately 10% of the total biomass is used for latex extraction and the remainder is either disposed or developed into other useful products and chemicals, such as fatty acid triglycerides, terpenes, sesquiterpenes, and waxes. Because latex is extracted primarily by a water-based process, the bagasse residual contains most of these useful compounds.

The resin-containing bagasse can be used without additional chemical processing. For example, it has been combined with a plastic binder to make high-density composite boards that are resistant to termite degradation. This bagasse could also be blended with other types of wood sources to make boards of intermediate density that will have the insect control properties. The bagasse can also be compressed into fireplace logs, briquettes, and worms or pellets for energy production. Such combustible material has higher energy content than other wood sources because of the resin, which can make up about 10% of the dry mass. Bagasse can also be converted into liquid fuel, and with improved pyrolysis technology, could become an economic source of diesel-type fuel. Deresinated bagasse could be a source of alcohol and other type of chemical entities for liquid fuel or solvents.

The resinous material can be solvent extracted from either the whole plant or the bagasse and can be used without purification. By impregnating wood with the crude resin extract, the wood can be protected against many types of wood destroying organisms. Guayule-based resin and epoxy polymers have been combined to make strippable coatings that can be used for storage protection of equipment. Plant improvement has increased rubber and biomass yields but at the same time changed the resin to rubber ratios from 1:1 to 2:1, and thus increases the resin component. Other Parthenium species with large biomass and resin content could provide a valuable source of raw material for natural wood preservatives. The future appears promising to develop existing and other species if the resin becomes an economically viable product.

Descriptions of current guayule cultivation practices have been reviewed extensively. Information in this regard can be found in, for example, Thompson and Ray (Breeding guayule. Plant Breed Rev. 6:93-165, 1989) and in Guayule Natural Rubber, edited by Whitworth and Whitehead (1991). Plant breeding has, and will continue to be, one of the most effective methods of increasing guayule productivity and quality. Varietal improvement of guayule, like many other crops, relies upon the propagation of superior strains of wild plants. However, variability within wild stands lowered yields, and this problem continued through the early attempts at cultivation because populations have been established with open-pollinated seeds collected from plants that were very heterogeneous genetically.

Thus a continuing goal of plant breeders is to develop stable high yielding guayule cultivars that are agronomically sound. The reasons for this goal are obvious to maximize the amount of biomass and/or latex and rubber produced per unit land area.

### Detailed Description of the Inventive Breeding Methods

Traditionally, the production of uniform hybrid varieties generally requires the development of homozygous inbred plants, followed by the crossing of these inbred plants, and the evaluation of the crosses. Plants that have been self-pollinated and selected for type over many generations become homozygous at almost all gene loci (i.e. near-isogenic) and produce a substantially uniform population of true breeding progeny, which is a homozygous plant. A cross between two such homozygous, near-isogenic plants of different varieties typically produces a uniform population of hybrid plants that display the same allelic heterozygosity at many loci.

In the case of guayule, perhaps the biggest challenge in developing commercial grade guayule hybrids through plant breeding is the highly complex reproduction biology of this crop plant which is associated with its facultative nature (i.e. apomixis and sexuality coexisting) and the high amount of heterozygosity in individual plants and the heterogeneous make-up of populations, results in the release of considerable variation whenever sexual reproduction (amphimixis) occurs. Most of the guayule varieties currently used in large-scale production are heterozygous at many loci and thus lack genetic uniformity. As such, a sexual cross between two genetically dissimilar parents typically results in a heterogeneous collection of F1 hybrids, with each individual plant from the same cross exhibiting a unique allelic segregation event. Applicants have observed not only genotypic segregation among F1 individuals, but also dramatic phenotypic differences. Further, the process of inbreeding of parent lines often time can be very consuming, especially because apomixis and sexuality coexist.

Although it has been assumed in guayule that apomixis assures genetic uniformity from generation to generation, its facultative nature (apomixis and sexuality coexisting) and the high amount of heterozygosity in individual plants and the heterogeneous make-up of populations, results in the release of considerable variation whenever sexual reproduction (amphimixis) occurs (Powers and Rollins, J. Amer. Soc. Agron., 37:96-112, 1945). Wild stands of guayule typically contain a natural polyploid series of diploids (2n=2x=36), triploids (2n=3x=54) and tetraploids (2n=4x=72); and under cultivation, individual plants have been identified with chromosome numbers up to octaploid (2n=8x=144). Diploids reproduce predominantly sexually, and polyploids reproduce by facultative apomixis. Guayule also has a sporophytic system of self-incompatibility and many plants contain B- or supernumerary chromosomes (Thompson and Ray, 1989, supra). Another reproductive feature is reported to occur frequently in polyploid guayule is haploidy, which is the reduction of chromosome number from, for example, 2N to 1N. In this instant, the egg cell has a reduced chromosome number because meiosis has occurred, but the stimulus for apomictic development still exists and the egg in the reduced condition produces a new haploid plant.

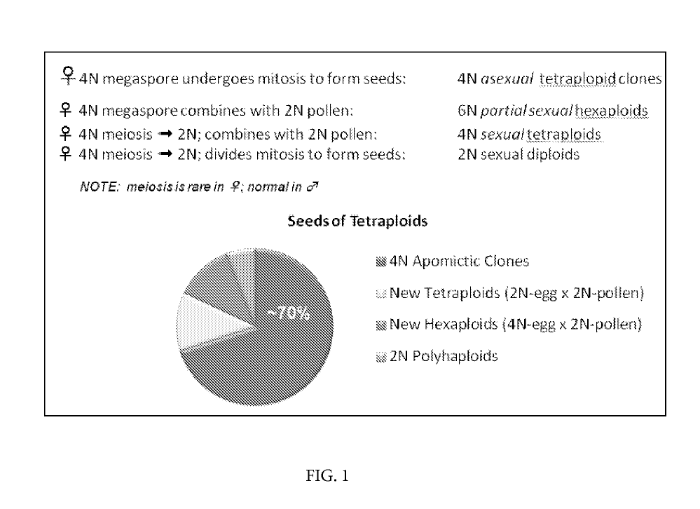

Due to the facultative nature of guayule, four classes of progeny generally exist. For example, as illustrated in FIG. 1, the origin and relative chromosome numbers of these four classes from tetraploid (2n=4x=72) parents illustrates the complexity of reproduction and the potential for release of genetic variability in this species. The predominant class of progeny arises from non-reduction of the megaspore mother cell (MMC), without fertilization. These are apomictic tetraploid progeny and are identical generically to the maternal parent. Progeny from fertilized, unreduced MMCs include plants with increased ploidy levels. In this example, these progeny would be hexaploid (2n=6x=108). Polyhaploid (2n=2x=36) plants are the result of meiotic reduction of the tetraploid MMC, and embryo development without fertilization. The final class would be amphimictic tetraploid (2n=4x=72) progeny that arises from normal reduction and fertilization. Thus, two reproductive modes produce tetraploid progeny, one by apomixis and the other by sexual reproduction. The remaining two progeny classes vary in chromosome number from the parental population.

In the past, attention has been primarily focused on tetraploid lines of guayule. The tetraploids are generally much larger and more vigorous than the diploids, and they readily produce seeds (clones) through apomixis (a marked advantage for rapid increase of a selected 4N genotype). Fields of a tetraploid line could be used to not only produce latex/rubber but could also be used for seed production. Breeders have performed mass selections on tetraploid lines to identify individuals with higher latex/rubber yields. These can be propagated by cuttings or by the apomictically-derived seeds. However, a problem with this approach is that only about 70% of the seeds from tetraploids breed true (FIG. 1). The other 30% is a combination of various off-types coming from the fertilization of the unreduced megaspore (4N), the fertilization of the reduced megaspore (2N), or the apomictically-derived seed coming from the reduced megaspore. This reduces the purity of the seed source leading to variation in plantation yields.

Triploids and pentaploids are known to occur in guayule. From a breeding point of view, these individuals are considered dead ends because they are male and female sterile. Although some have been documented as having a relatively high latex content, these were generally not pursued for commercial latex production because a field of only triploids or pentaploids, although productive in terms of latex, may have been unproductive in terms of seeds for subsequent plantation expansion. In one embodiment of the present invention, Applicants further contemplate that apomictic seed production in triploids and pentaploids may require the presence of fertile plants to assure fertilization of the polar nuclei and subsequent development of the seed endosperm.

The breeding methods disclosed herein differ fundamentally from current breeding methods described above. Most existing guayule germplasm consists of apomictically reproducing tetraploid accessions, which have received most of the attention in breeding programs. In contrast, sexually reproducing, largely self-incompatible diploids, have had only limited use in current guayule breeding programs. As described in greater details in the Examples below, Applicants have developed a breeding strategy that takes advantage of all these subtle reproductive nuances in guayule, and combines them into a breeding method that permits the rapid development of productive hybrid triploid and pentaploid guayule plants.

In principle, the breeding methods according to the present invention can be applied to any apomictic plant species. In particular, the presently disclosed breeding methods are preferably used with apomictic plants that are important or interesting for agriculture, horticulture, for the production of biomass used in producing latex, liquid fuel molecules, and other chemicals, and/or forestry.

Thus, the invention has use over a broad range of plants, preferably higher plants pertaining to the families of Asteraceae, Orchidaceae, Poaceae, and Rosaceae. Plants of the genera Agropyrum, Allium, Amelanchier, Antennaria, Beta, Boechera, Brachiaria, Cenchrus, Chloris, Compositae, Coprosma, Cortaderia, Crataegus, Cytrus, Datura, Dichanthium, Eragrostis, Erigeron, Eriochloa, Eupatorium, Heteropogon, Hieracium, Hyparrhenia, Hypericum, Ixeris, Panicum, Parthenium, Paspalum, Paspalum, Pennisetum, Poa, Ranunculus, Rubus, Sorghum, Taraxacum, Themeda, Tripsacum, and Urochloa are particularly suitable.

Particularly suitable species include members of the genus Parthenium, especially Parthenium argentatum.

### A. Vegetative Propagation, Tissue Culture, and In Vitro Regeneration of Guayule Plants

A further aspect of the present invention relates to tissue cultures and vegetative regeneration of the guayule plants provided herein. As used herein, the term “tissue culture” indicates a composition comprising isolated regenerable cells or protoplasts of the same or a different type or a collection of such cells organized into parts of a plant. Exemplary types of tissue cultures are embryo, protoplast, meristematic cell, callus, pollen, glume, panicle leaf, pollen, ovule, cotyledon, hypocotyl, root, root tip, pistil, anther, floret, seed, stalk and rachis, and the like. As used herein, the term “plant” in reference to a plant tissue culture includes plant cells, plant protoplasts, plant cells of tissue culture from which guayule plants can be regenerated, plant calli, plant clumps, and plant cells that are intact in plants or parts of plants.

Means, materials, systems, and methods for preparing and maintaining plant tissue culture are well known in the art. Technical information, systems, materials, and methods proven to useful in plant tissue cultures can also be found in, e.g., Komatsuda T., et al., Crop Sci., 31:333-337 (1991); Stephens P A et al., Theor. Appl. Genet., 82:633-635 (1991); Komatsuda T. et al., Plant Cell, Tissue and Organ Culture, 28:103-113, 1992; Dhir S. et al., Plant Cell Reports, 11:285-289 (1992); Pandey P. et al., Japan J. Breed., 42:1-5 (1992); and Shetty K et al., Plant Science, 81:245-251 (1992); as well as U.S. Pat. Nos. 5,024,944; and 5,008,200.

Further, tissue culture of various tissues of guayule and regeneration of guayule plants therefrom is well known and widely published. For example, information in this regard can be found in Radin et al. Plant Sci. Letters, Vol. 26:2-3, pp. 301-310, August 1982; Norton et al., Phytochemistry, Vol. 30:8, pp. 2611-2614, 1991; Castillon and Komish, In Vitro Cell. Dev. Biol.-Plant, Vol. 36:3, pp 215-219, 2000; which describe certain common tissue culture techniques used to regenerate guayule plantlets.

Thus, another aspect of this invention is to provide cells and tissues which upon growth and differentiation produce guayule plants having the physiological and morphological characteristics of a guayule hybrid plant disclosed herein.

### B. Production of Double Haploids

The production of double haploids can also be used for the development of plants with a homozygous phenotype in the breeding program. For example, a guayule plant disclosed herein as a parent can be used to produce double haploid plants. Double haploids are produced by the doubling of a set of chromosomes (1N) from a heterozygous plant to produce a completely homozygous individual. A number of methods useful for doubling chromosome number are known. Information in this regarding can be found in, for example, M. Maluszynski et al., Doubled Haploid Production in Crop Plants: A Manual, Kluwer Acad. Publishers, 2003; Wan, et al., “Efficient Production of Doubled Haploid Plants Through Colchicine Treatment of Anther-Derived Maize Callus,” Theoretical and Applied Genetics, 77:889-892 (1989), and U.S. Pat. No. 7,135,615. Additional information for materials, systems, and methods useful for the production of double haploids can be found in, for example, Bossoutrot and Hosemans, Plant Cell Reports, 4:300-303, 1983; Chen et al., Plant Breeding, 113:217-221, 1994; Liu et al., Plant Cell Reports, 24:133-144, 2001. This technique can be advantageous because the process omits the generation of selfing needed to obtain a homozygous plant from a heterologous source.

Methods for obtaining haploid plants have been disclosed in, e.g., Kobayashi M., et al., Journal of Heredity, 71(1):9-14 (1980); Pollacsek M., 12(3):247-251, Agronomie, Paris (1992); Cho-Un-Haing et al., Journal of Plant Biol., 39(3):185-188 (1996); Verdoodt L., et al., 96(2):294-300 (February 1998); Genetic Manipulation in Plant Breeding, Proceedings International Symposium Organized by EUCARPIA, Berlin, Germany (Sep. 8-13, 1985); Thomas W J K, et al., “Doubled haploids in breeding,” in Doubled Haploid Production in Crop Plants, Maluszynski, M., et al. (Eds.), Dordrecht, The Netherland Kluwer Academic Publishers, pp. 337-349 (2003).

Haploid induction systems have also been developed for various plants to produce haploid tissues, plants and seeds. The haploid induction system can produce haploid plants from any genotype by crossing a selected line (as female) with an inducer line. Such inducer lines for maize include Stock 6 (Coe, 1959, Am. Nat. 93:381-382; Sharkar and Coe, 1966, Genetics 54:453-464), KEMS (Deimling, Roeber, and Geiger, 1997, Vortr. Pflanzenzuchtg 38:203-224), or KMS and ZMS (Chalyk, Bylich & Chebotar, 1994, MNL 68:47; Chalyk & Chebotar, 2000, Plant Breeding 119:363-364), and indeterminate gametophyte (ig) mutation (Kermicle 1969 Science 166:1422-1424).

Thus, according to one aspect of the invention, there is provided a process for making a progeny guayule plant substantially homozygous to a guayule hybrid plant disclosed herein by applying double haploid methods to the guayule hybrid plant or to any successive filial generation. Based on studies in maize, and more recently in switchgrass, such methods would decrease the number of generations required to produce a variety with similar genetics or characteristics to the starting guayule hybrid of the invention. See Bernardo and Kahler, Theor. Appl. Genet. 102:986-992, 2001. Additionally, upon review of the present disclosure, the artisan skilled in the art will immediately appreciate that the presently disclosed doubled-haploid guayule plants can be used to generate parental lines in the production of, for example, substantially uniform apomictic triploids and pentaploids.

Descriptions of other breeding methods that are commonly used for different traits and crops can be found in one of several reference books (e.g., Allard, 1960; Simmonds, 1979; Sneep et al., 1979; and Fehr, 1987).

### C. New Guayule Plants Derived by Genetic Engineering

The advent of new molecular biological techniques has allowed the isolation and characterization of genetic elements with specific functions, such as sequences encoding specific protein products or sequences having promoter activity. Scientists in the field of plant biology developed a strong interest in engineering the genome of plants to contain and express foreign genetic elements, or additional, or modified versions of native or endogenous genetic elements in order to alter the traits of a plant in a specific manner. Any heterologous DNA sequences, whether from a different species or from the same species which are inserted into the genome using genetic transformation, are referred to herein collectively as “transgenes”.

Plant transformation involves the construction of an expression vector which will function in plant cells. Such a vector comprises DNA comprising a gene under control of or operatively linked to a regulatory element (for example, a promoter). The expression vector may contain one or more such operably linked gene/regulatory element combinations. The vector(s) may be in the form of a plasmid, and can be used alone or in combination with other plasmids, to provide transformed guayule plants, using transformation methods as described below to incorporate transgenes into the genetic material of the guayule plant(s).

In some embodiments of the invention, a transgenic variant of a guayule hybrid plant disclosed herein may contain at least one transgene but could contain at least 1, 2, 3, 4, 5, 6, 7, 8, 9, 10 transgenes and/or no more than 15, 14, 13, 12, 11, 10, 9, 8, 7, 6, 5, 4, 3, or 2 transgenes.

In one embodiment of the invention, various genetic elements can be introduced into the plant genome using transformation techniques. These elements include, but are not limited to genes, coding sequences, inducible, constitutive, and tissue specific promoters, enhancing sequences, and signal and targeting sequences. For example, see the traits, genes and transformation methods listed in Pan et al., Plant Cell, Tissue and Organ Culture, 46:2, 143-150, 1996; Veatch et al., Ind. Crop Prod., 22:65-74. 2005; Dong et al., Plant Cell Rep., 25:1, 26-34, 2006; and U.S. Pat. No. 8,013,213.

In one embodiment, there is provided a process for producing a guayule plant that further comprises a desired trait. The process comprises transforming a guayule plant provided herein with a transgene that confers a desired trait. Another embodiment of the invention comprises a transformed guayule plant produced by this process, and seeds produced by such transformed plants. In yet another embodiment, the desired trait may be one or more of high productivity, high latex yield, high resin yield, high overall rubber yield, abiotic stress tolerance, biotic stress tolerance, disease resistance, improved water use efficiency, improved nitrogen use efficiency, and combinations of any thereof. The specific genes useful for this process may be any gene known in the art for its ability to confer such traits. Examples of such trait genes include, but not limited to genes encoding various allylic diphosphate synthases in the rubber biosynthesis pathway, including geranylgeranyl pyrophosphate synthase (GGPP); hexa-heptaprenyl pyrophosphate synthase, and farnesyl pyrophosphate synthase (FPP) (U.S. Pat. No. 8,013,213); Veatch et al., Ind. Crop Prod., 22:65-74. 2005; Dong et al., Plant Cell Rep., 25:1, 26-34, 2006.

Also provided in certain embodiments of the present invention are seeds, plants, plant cells and plant parts disclosed herein further comprising a transgene.

A number of methods for plant transformation, which have been previously developed for the genetic transformation of various plant species, can be deployed for the transformation of guayule. See, for example, Mild et al., “Procedures for Introducing Foreign DNA into Plants” in Methods in Plant Molecular Biology and Biotechnology, Glick B. R. and Thompson, J. E. Eds. (CRC Press, Inc., Boca Raton, 1993) pages 67-88. In addition, expression vectors and in vitro culture methods for plant cell or tissue transformation and regeneration of plants are readily available. See, for example, Gruber et al., “Vectors for Plant Transformation” in Methods in Plant Molecular Biology and Biotechnology, Glick B. R. and Thompson, J. E. Eds. (CRC Press, Inc., Boca Raton, 1993) pp. 89-119. Suitable genetic transformation methods include electroporation (U.S. Pat. No. 5,384,253), micro-projectile bombardment (Sanford I., Part. Sci. Technol. 5:27, 1987; Sanford J. C., Trends Biotech. 6:299, 1988; Klein et al., BioTechnology 6:559-563, 1988; Sanford, J. C., Physiol Plant 7:206, 1990; Klein, et al., Biotechnology 10:268, 1992; U.S. Pat. Nos. 5,550,318; 5,736,369; 5,538,880; and PCT Patent Pub. No. WO 95/06128), Agrobacterium-mediated transformation (Horsch et al., Science 227:1229, 1985; Kado, Crit. Rev. Plant Sci. 10:1, 1991; Moloney, et al., Plant Cell Reports 8:238, 1989; U.S. Pat. Nos. 5,563,055; 5,591,616; and EP Pat. Pub EP672752), direct DNA uptake transformation of protoplasts (Omirulleh et al., Plant Mol. Biol., 21(3):415-428, 1993), and silicon carbide fiber-mediated transformation (U.S. Pat. Nos. 5,302,532 and 5,464,765).

More specifically, methods for the genetic transformation of guayule are known to those of skill in the art. See, e.g., U.S. Pat. No. 8,013,213); Veatch et al., Ind. Crop Prod., 22:65-74. 2005; Dong et al., Plant Cell Rep., 25(1):26-34, 2006. Transformed seeds, plants, plant cells, and plant parts obtained by such transformation methods are intended to be within the scope of this invention. Following transformation of guayule target tissues, expression of a suitable selectable marker gene allows for preferential selection of transformed cells, tissues and/or plants, using regeneration and selection methods well-known in the art. Each of the above references is incorporated herein by reference in its entirety.

Additional technical details related to materials, systems and methods useful for genetic transformation of guayule, including selectable markers, suitable promoters, and expression vectors, have been previously documented in, e.g., Li et al., Plant Cell Tissue Organ Cult. 92:173-181, 2008; Khemkladngoen et al., Plant Biotechnol. Rep. 5:235-243, 2011; Kumar et al., Ind. Crops Prod. 32:41-47, 2010; and Tsuchimoto et al., Plant Biotechnol. 29:137-143, 2012; US Pat. Pub. Nos. US20060217512 and US20060218660, each of which is incorporated herein by reference in its entirety.

It is understood to those of skill in the art that a transgene need not be directly transformed into a plant, as techniques for the production of stably transformed guayule plants that pass single loci to progeny by Mendelian inheritance is well known in the art. Such loci may therefore be passed from parent plant to progeny plants by standard plant breeding techniques that are well known in the art. Thus, the foregoing methods for transformation would typically be used for producing a transgenic variety. The transgenic variety could then be crossed with another (non-transformed or transformed) variety, in order to produce a new transgenic variety. Alternatively, a genetic trait which has been engineered into a particular guayule variety using the foregoing transformation techniques could be moved into another variety using traditional backcrossing techniques that are well-known in the plant breeding arts. For example, a backcrossing approach could be used to move an engineered trait from a public, non-elite variety into an elite variety, or from a variety containing a foreign gene in its genome into a variety or varieties which do not contain that gene.

### D. Methods of Producing Plant-Derived Products

Also provided herein are methods of producing biomass or at least one plant-derived product by obtaining a hybrid plant disclosed herein, or a part thereof, followed by producing the biomass or at least one plant-derived product. Descriptions of current guayule cultivation practices have been reviewed extensively. Information in this regard can be found in, for example, Thompson and Ray (Breeding guayule. Plant Breed Rev. 6:93-165, 1989) and in Guayule Natural Rubber, edited by Whitworth and Whitehead (1991).

In some embodiments, products such as latex, resin, fatty acid triglycerides, terpenes, sesquiterpenes, or waxes can be recovered from the hybrid plants of the invention by recovery means known to those skilled in the art. Methods and systems useful for the production of resins derived from plant species bearing rubber and rubber-like hydrocarbons have been previously reported. In addition, methods and systems useful for preparation and utilization of multi-component copolymers of guayule resin with improved physical and chemical properties are also well documented. Information in this regard can be found in, for example, Ray, D. T. 1993. Guayule: A source of natural rubber. p. 338-343. In: J. Janick and J. E. Simon (eds.), New Crops. Wiley, New York; Ray et al., Industrial Crops Products, 22:15-25, 2005; Veatch et al., Ind. Crop Prod., 22:65-74. 2005; Dong et al., Plant Cell Rep., 25:1, 26-34, 2006; Estilai et al., Developing guayule as a domestic rubber crop, California Agriculture, Sep.-Oct. 29-30, 1988; U.S. Pat. Pub. Nos. US20060217512, US20090099309, US20060218660, US20090163689, PCT Pat. Pub. Nos. WO2007081376, WO2007136364, and WO2008147439, U.S. Pat. Nos. 5,580,942; 5,717,050; 7,259,231; 7,790,036; and 8,013,213; each of which is incorporated herein by reference in its entirety.

The discussion of the general methods given herein is intended for illustrative purposes only. Other alternative methods and embodiments will be apparent to those skilled in the art upon review of this disclosure. The following examples are offered to illustrate, but not limit, the invention.

A number of embodiments of the invention have been described. Nevertheless, it will be understood that elements of the embodiments described herein can be combined to make additional embodiments and various modifications may be made without departing from the spirit and scope of the invention. Accordingly, other embodiments, alternatives and equivalents are within the scope of the invention and claimed herein. Headings within the applications are solely for the convenience of the reader, and do not limit in any way the scope of the invention or its embodiments.

All publications and patent applications mentioned in this specification are herein incorporated by reference to the same extent as if each individual publication or patent application was specifically can individually indicated to be incorporated by reference.

The following examples are included merely for the purposes of illustration of certain aspects and embodiments of the present invention, and should not be construed as limiting the invention in any way. The materials and methods employed in the examples below are for illustrative purposes, and are not intended to limit the practice of the present invention thereto. Any materials and methods similar or equivalent to those described herein can be used in the practice or testing of the present invention. Other alternative methods and embodiments will be apparent to those of skill in the art upon review of this disclosure.

Most guayule germplasm today consists of apomictically reproducing tetraploid accessions, which have received most of the attention in breeding programs. Sexually reproducing, largely self-incompatible diploids, have had only limited use in current guayule breeding programs. Applicants have developed a breeding strategy that takes advantage of these subtle reproductive nuances in guayule, and combines them into a breeding method that permits the rapid development of productive hybrid triploid and pentaploid guayule plants.

### EXAMPLES

### Example 1

### Identify High Yielding Apomictically-Reproducing Triploid and Pentaploid Guayule from Existing Production and Cultivation Fields

10,000 two-year-old guayule plants were randomly tagged in a rubber production field in Coolidge, Ariz. Seed was collected from each tagged plant individually to create a seed lot. Each seed lot was then cleaned and germinated. After the seedlings were large enough to survive sampling, two 5 mm hole punches were sampled from three seedlings from each seed lot in triplicate and placed into a 96 well microtube rack. If three seedlings did not germinate, the seed lot was not analyzed. Tissue was also collected from diploid, triploid, and tetraploid intraspecific control plants and loaded on the microtube rack. A 3 mm carbide bead and 500 μL of Baranyi I solution were loaded into each well before disrupting the samples with a TissueLyser II at 27 Hz for 30 seconds. Nuclear lysates were then centrifuged through a 30-40 um filter to remove cellular debris. Two parts Baranyi II solution mixed with SYBR Green I were added to one part of the nuclei extract to bring the final sample to 2×SYBR Green I and a neutral pH (7.0-8.0). Stained nuclei samples were allowed to incubate in the dark at room temperature for 30 minutes before analysis. The fluorescence value relative to the DNA content of nuclei for each sample was acquired using an Attune Flow Cytometer and Attune Autosampler. An acquisition flow rate of 100 μl/min and an acquisition volume of 50 μl were used. Nuclei peaks were manually gated and resulting median fluorescence values compared against intraspecific controls to generate ploidy calls.

If the seedlings were found to be triploid or pentaploid, phenotypic measurements were taken of the field plant, and it was harvested for rubber analysis via Accelerated Solvent Extraction (ASE). The plant was cut approximately 4 inches above ground level, defoliated, and several cuttings were taken for propagation. The mass of the plant material was recorded before and after defoliation. Additionally, the root and stem material below the cut height were extracted and transplanted to a 3 gallon pot for propagation. The defoliated material was transported in a cooled environment to a processing facility where it was homogenized with a chipper shredder and mixed. A 100 g aliquot of this material was passed through a Wiley Mill to create a uniform particle size.

After passing through the Wiley Mill, a 2 g aliquot of the sample was removed for moisture analysis. The mass of this aliquot was recorded, and the sample was dried for 12 hours at 80° C. after which the mass was recorded again. The moisture content of the plant and subsequently the dry mass of the plant were calculated from these values. The remaining sample was submerged in a 1 g/L Bostex 517/24 AO solution for 10 minutes. The soaked sample was then placed in several layers of cheesecloth and wrung to remove excess AO solution. Following ten minutes of treatment, the sample was filtered of liquid antioxidant and placed in a vacuum-sealed bag for shipment on ice to the Chemical Analytics facility.

Three replicate samples containing four grams each of the homogenized biomass were sampled, dried for 24 hours at 45° C., and then ground with a mill to achieve a finer particle size. Approximately 0.5 grams of the finely ground biomass was sampled, mixed with sand, and then loaded into an ASE Cell. The replicate biomass samples for each plant were extracted on a Dionex ASE 350 with hexane. The following were the ASE parameters used: Temperature: 40° C.; Static Time: 5 minutes; Number of cycles: 9; Rinse Volume: 150%; Purge Time: 60 seconds; Cell type: 10 mL (sst); Solvent Saver: off. The resulting hexane extracts were centrifuged at 2500 g for 15 minutes to pellet contaminants. The supernatant of each extract was poured off into its own aluminum tray and allowed to completely evaporate. The mass of the extracted rubber was measured and then divided by the dry mass of the sample loaded into the ASE cell to generate the percent rubber content.

From the measured percent rubber, the defoliated mass of the plant, and the measured moisture content, the total rubber yield of the plant could be calculated. If the sample was found to be high yielding (either total rubber by dry weight or percent rubber), all seedlings from the seed lot and cuttings from the field plant were transplanted to 3 gallon pots. The ploidy of all seedlings, cuttings, and field plants was confirmed. A plant was progressed if at least five plants from the seed lot existed, and at least 80% of the plants tested positive for a ploidy of 3N or 5N. Seed was then bulked from these plants as well as the original field plant to produce a high yielding triploid or pentaploid seed lot.

From the 10,000 seed lots cleaned and germinated, 3,100 produced a minimum of three seedlings that were analyzed for ploidy. Of these, 846 seed lots were determined to be triploid or pentaploid, with more than two triploid or pentaploid seedlings of the three tested. 562 of these plants were harvested and analyzed for rubber content with ASE. 144 of these seed lots have been advanced for seed multiplication based upon a rubber content ranging between 6 to 9.5% and ploidy of 3N and 5N.

This methodology can be used to rapidly discover high yielding triploid and pentaploid plants in an existing field. The seed from these plants can then be germinated, multiplied, and bulked to quickly produce a high yielding, uniform triploid or pentaploid seed lot. The collection of phenotypic data at time of harvest allows the breeder to predict the behavior of its progeny and incorporate this information to determine which lines should be advanced.

Most of the guayule varieties currently used in large-scale planting are heterozygous at many loci and thus lack genetic uniformity. As such, a sexual cross between two genetically dissimilar parents typically results in a heterogeneous collection of F1 hybrids, with each individual plant from the same cross exhibiting a unique allelic segregation event. Applicants have observed not only genotypic segregation among F1 individuals, but also dramatic phenotypic differences. Therefore, since the identification of suitable parental plants is generally considered one of the most important points in any breeding program, Applicants have initiated a large scale screening of guayule germplasm collection for plants with different levels of ploidy. Specifically, the identification of apomictic plants useful for the methods of the invention can be achieved by progeny testing open pollinated seed from selected plants. Since a number of size features of guayule plants, such as size of the fruit complex and the trichome structures on leaf surface, are tightly correlated with chromosome numbers and the plants' ploidy, morphologically variable progeny from a plant can be scored and used as an indication of a plant's sexual origin. The frequency of uniform or maternal progeny from a plant would indicate the level of apomictic reproduction. Applicants are analyzing at least 10 to 25 progeny individuals of each tested plant to obtain a reliable estimate of the plant's reproductive behavior. This identification step is especially important because guayule reproduces by facultative apomixis.

Applicants also contemplate conducting cytological analyses coupled with chromosome staining techniques, which are generally more rapid and more scalable than progeny testing, for determining the method of reproduction of a given plant. Readily available for this purpose are a number of ovule-clearing techniques that allow one to classify the reproductive behavior of a plant within 2 or 3 days after collecting the ovaries. Applicants plan to collect a few flowers at the beginning of anthesis and to classify the reproductive behavior of the plant before it completes anthesis. Apospory and adventitious embryony are the apomictic mechanisms that can be conveniently identified at anthesis. In some instances, apospory can be identified by the presence of multiple embryo sacs, the lack of antipodal development and shape and orientation of embryo sacs in the ovule. Adventitious embryony can also be identified because the embryo develops as a bud-like structure through mitotic division of somatic cells of the ovule, integuments or ovary wall. Diplospory can also be identified by cytological examinations at earlier stages of ovule development. Lack of meiosis or a linear tetrad of megaspores is an indication for diplospory. In addition, the lack of fluorescing callose in the walls of dyads, tetrads and megaspore mother cells is also an indication for diplospory.

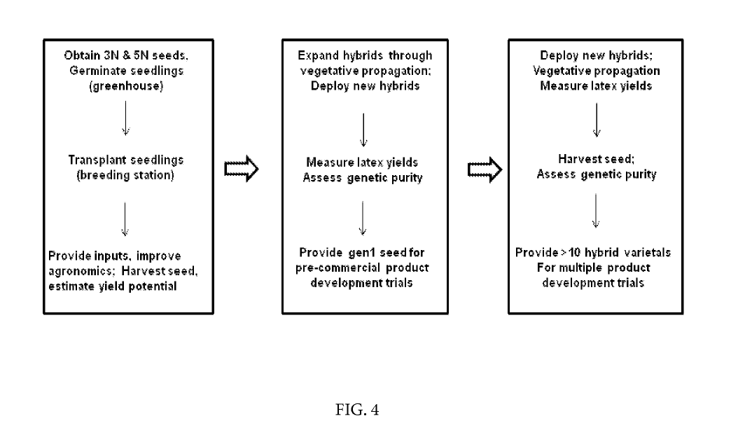

Among guayule plants with different levels of ploidy, genotypes with diploid (2N), tetraploid (4N), and hexaploid (6N) genome are selected and used as parental lines for interploidy mating phases of the presently disclosed breeding scheme (Example 2). Genotypes with triploid 3N or pentaploid 5N genomes are selected for the development of pre-commercial hybrids, as described in greater details in, e.g. FIG. 4. In some instances, the guayule genotypes selected as described above are intercrossed with one another, followed by progeny testing to evaluate general combining abilities (intercrossability).

### Example 2

### Diploid and Tetraploid Guayule Plants Serve as Female and Male Parents, Respectively, for Production of Guayule Hybrids

An experiment was performed in order to confirm that harvested seeds from a diploid Parthenium argentatum (guayule) plant, when crossed with a tetraploid guayule, produced triploid F1 hybrid offspring. Three tetraploid and three diploid guayule plants were pruned of flowers. Each diploid plant was placed in a cage with one of the tetraploid plants. Flies were added to the cage weekly to facilitate pollination. Achenes were harvested from each diploid plant after thirty days of isolation. The achenes were then germinated in a growth chamber with a 14 hour day length, day temperature of 27° C. and night temperature of 22° C. and transferred to 4″ pots and grown until the plants were fully established. Next, two 5 mm leaf punches were collected in triplicate along with leaf samples from known diploid, triploid, and tetraploid control plants. The tissue samples were disrupted with a 3 mm carbide bead and 500 μL of Baranyi I solution in 96 well format TissueLyser at 27 Hz for 30 seconds. Nuclear lysates were then centrifuged through a 30-40 μm filter to remove cellular debris. Two parts Baranyi II solution mixed with SYBR Green I were added to one part of the nuclei extract to bring the final sample to 2×SYBR Green I and a neutral pH (7.0-8.0). Stained nuclei samples were allowed to incubate in the dark at room temperature for 30 minutes before analysis. The fluorescence value relative to the DNA content of nuclei for each sample was acquired using an Attune Flow Cytometer and Attune Autosampler and compared to control plants in order to assign a ploidy value to the seedlings from which the samples were collected. The results are summarized in Table 1 below. Note that the ploidy level of all the F1 seedlings derived from seeds collected from the three diploid plants individually crossed with tetraploid plants resulted in 100% triploid (F1 hybrid) offspring, thus confirming the ability to effectively produce triploid hybrid seeds in guayule by selectively crossing a diploid guayule with a tetraploid guayule. The self incompatible nature of the diploid guayule effectively limits the production of diploid progeny and biases production to triploid progeny. This demonstrates an effective method for producing commercial hybrid seed in guayule.

TABLE 1
Ploidy calls for the F1 seedlings of each caged cross.

| | F1 Seedlings | | | | |
|---|---|---|---|---|---|
| | Diploid, | Triploid, | Tetraploid, | | % |
| Location | 2X | 3X | 4X | Total | Hybrid |
| CAGE 1 | 0 | 7 | 0 | 7 | 100% |
| CAGE 2 | 0 | 16 | 0 | 16 | 100% |
| CAGE 3 | 0 | 20 | 0 | 20 | 100% |

The results from the next experiment support the claim that seeds derived from triploid hybrid plants produce clonal triploid hybrid seeds with the same genetic constitution of the parental triploid hybrid plant. In this experiment, leaf punches were collected from twelve plants each originating from a single hybrid obtained from a single triploid hybrid plant seed lot. DNA was extracted using a modified protocol with the PureLink Genomic Plant DNA Purification Kit from Life Technologies and the samples were submitted to Genotyping-bySequencing (GBS), performed at the Genomic Diversity Facility at Cornell University. Sequencing data was analyzed with the UNEAK pipeline of the TASSELi package, outputting SNP calls in Variant Call Format (VCF) for the submitted DNA samples. The resulting SNPs were filtered with custom Perl scripts in conjunction with VCFtoolsii according to the following parameters to obtain a set of high quality SNP calls to evaluate genetic similarity: a) Genotype Quality >98; b) Individual Missingness <98%; c) SNP Callrate >1%; d) Minor Allele Frequency ≧ 0.01.

Additionally, SNPs were filtered to include only homozygous SNP calls to avoid shortcomings of the GBS method of genotyping. The genetic distance between these individuals was then calculated according to Identity-by-State (IBS) using the TASSEL package, allowing for four comparisons at each SNP site that is shared between two individuals.

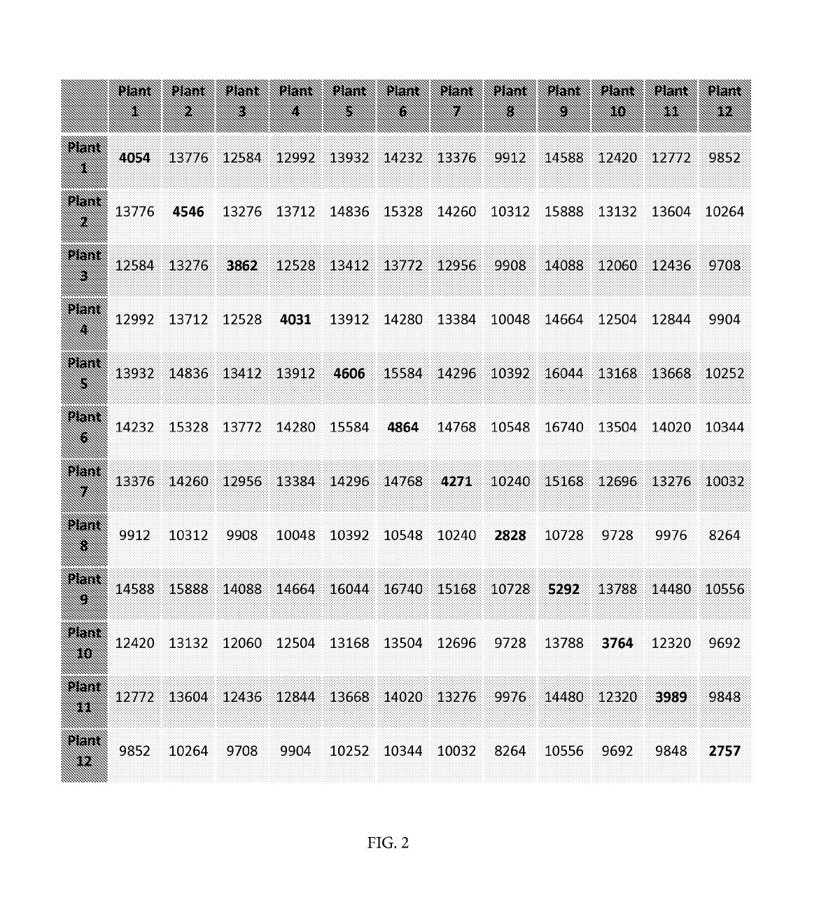

An average of 3,150 SNP sites were compared between each pair of individuals. The values used to compute genetic distance are shown in FIG. 2. Genetic distance is calculated as:

$$Genetic Distance = \frac{Total Comparison - Individual Comparison}{Total Comparison}$$

Genetic distance between all individual in this experiment is 0.0000. Average genetic distance of these plants to other guayule samples analyzed with GBS is 0.0448. FIG. 2 depicts the results from the experiment.

Note that no genetic differences were detected in twelve plants grown from seed and originating from a single hybrid seed lot when comparing several thousand SNP sites. This provides significant confidence that seedlings produced from triploid hybrids are produced apomictically and will replicate the seed donor's genetics, thus providing an effective method by which commercial seed production from any selected triploid hybrid progeny can be rapidly scaled to produce uniform hybrid progeny.

In the next experiment, total rubber content of clonal triploid hybrid plants was compared. Three plants were randomly selected from an 18-month-old accession of triploid hybrid plants grown in the field. For each plant all biomass 4 inches above the ground was harvested between 8 am-9 am. The biomass was defoliated, cut into smaller pieces, and processed for one minute in a Waring blender to reduce particle size. The blended sample was then submerged in a 1 g/L Bostex 517/24 AO solution for 10 minutes. The soaked sample was then placed in several layers of cheesecloth and wrung to remove excess AO solution. Three replicate samples containing four grams each of blended biomass were sampled, dried for 24 hours at 45° C., and then ground with dry ice in a smaller blender to achieve a finer particle size. Approximately 2.5 grams of the finely ground biomass was sampled, mixed with sand, and then loaded into an ASE cell. The replicate biomass samples for each plant were extracted on a Dionex ASE 350 first with acetone and then with hexane. The following were the ASE parameters used: Temperature: 40° C.; Static Time: 5 minutes; Number of cycles: 7; Rinse Volume: 150%; Purge Time: 60 seconds; Cell type: 10 mL (sst); Solvent Saver: off. The resulting acetone and hexane extracts were poured off into their own aluminum trays and allowed to completely evaporate. The mass of the extracted rubber and resins were measured and then divided by the dry mass of the sample loaded into the ASE cell to generate the percent rubber content. The total resin & rubber content of the plants derived from the ASE analysis are depicted below in Table 2. Note that in all cases, rubber and resin contents were all very similar, demonstrating that triploid clonal progeny derived from the apomictically produced seeds of a triploid hybrid plant, when grown under field conditions, produce comparable levels of physiologically derived product; in this case resin and rubber. This further demonstrates the commercial value (uniformity of resin and rubber yield) of using apomictically derived seeds of triploid hybrid plants to produce a uniform product.

TABLE 2
Percent rubber and resin values in three triploid hybrid plants.

| Plant ID/ | % Extract in | Mean Extract | |
|---|---|---|---|
| Extract | each sample | in Sample (%) | % RSD |
| Plant 9 | 6.0554 | 6.2212 | 3.2358 |
| (Resin) | 6.4452 | | |
| | 6.1629 | | |
| Plant 10 | 6.882 | 6.827 | 1.4019 |
| (Resin) | 6.8825 | | |
| | 6.7165 | | |
| Plant 11 | 5.9455 | 6.3373 | 10.7713 |
| (Resin) | 7.1255 | | |
| | 5.9408 | | |
| Plant 9 | 4.1114 | 4.2878 | 3.5947 |
| (Rubber) | 4.3966 | | |
| | 4.3554 | | |
| Plant 10 | 4.5523 | 4.539 | 0.7224 |
| (Rubber) | 4.5631 | | |
| | 4.5017 | | |
| Plant 11 | 4.2126 | 4.5578 | 11.0097 |
| (Rubber) | 5.1335 | | |
| | 4.3274 | | |

The plants were also subjected to the GBS DNA fingerprinting method described in Experiment 2 to verify clonality with data tabulated in Table 3 below:

TABLE 3
Values resulting from IBS analysis of SNP calls.

| | Plant 9 | Plant 10 | Plant 11 |
|---|---|---|---|
| Plant 9 | 5292 | 13788 | 14480 |
| Plant 10 | 13788 | 3764 | 12320 |
| Plant 11 | 14480 | 12320 | 3989 |

The values along the diagonal (bolded) indicate the number of SNP sites with genotype calls passing the filters applied to the dataset for each individual. The values below the diagonal indicate the total number of IBS comparisons per pair of individuals (4×total common SNPs). The values above the diagonal indicate the number of these comparisons that were identical between the two individuals. In summary, the three plants tested exhibited very similar rubber contents (<0.3% difference) and identical SNPs.

As discussed above, diploid guayule plants are sexually fertile but largely self-incompatible sexually reproducing diploids (2n=36), and therefore have had only limited use in previously reported guayule breeding programs. Meiosis has been reported previously to occur normally in guayule diploid plants with the formation of 1N egg cell and 1N pollen. When a large number of diploid hybrid plants were planted and phenotypically characterized, Applicants observed approximately <5% inbreeding. Moreover as described above, when 2N plants are pollinated with pollen from tetraploid 4N plants, only triploid 3N hybrid seeds are produced.

Thus, according to one aspect of the presently disclosed breeding methods, when sexually fertile diploid plants are used as female parents to receive pollen from sexually productive tetraploid plant, thus producing sexually derived triploid progeny seed that is formed on the diploid parent. The resulting triploid hybrid seeds are planted, and further evaluated to identify the most productive individuals (i.e., producing high latex, rubber, or resin) identified through standard assays. Those individuals can then be used to produce clonal propagants via rooted cuttings, tissue culture or apomictic triploid seed. This then becomes the seed production source from which seeds are obtained for planting highly productive plantations of uniform triploid guayule.

In some preferred embodiments of the presently disclosed plant breeding methods interploidy crosses are performed between highly productive diploid female parents and tetraploid male parents that are genetically distinct, so as to potentially maximize the hybrid vigor (heterosis) among the resulting progeny.

In other preferred embodiments, interploidy crosses are performed between highly productive diploid female parents and tetraploid male parents that are genetically distinct from each other and that are inbred or essentially homozygous lines, such that all the resulting F1 hybrid triploid progeny are identical and highly productive. In this case, the parental lines themselves are used as the seed production material for generating highly productive, uniform F1 hybrids triploids for plantation development.

In a further embodiment of the invention, highly productive tetraploids used as female parents are fertilized with pollen from highly productive hexaploids as male parents to obtain rare pentaploid and heptaploid progeny formed on the tetraploid female parents. The pentaploid and heptaploid progeny are subsequently screened to identify the highly productive individuals that are then be clonally propagated via cuttings, vegetative production, and apomictically derived seeds. Because these plants have a higher ploidy number, the expectation is that they will be producing more latex, rubber or resin than one would find in tetraploids, triploids or diploids. The resulting pentaploid and heptaploid hybrid seeds are planted, and further evaluated to identify the most productive individuals (high latex, rubber or resin) identified through standard assays. Those individuals can then be used to produce clonal propagants via rooted cuttings, tissue culture or apomictically produced seeds.

### Example 3

### Improve Parental Lines Through Trait Introgression

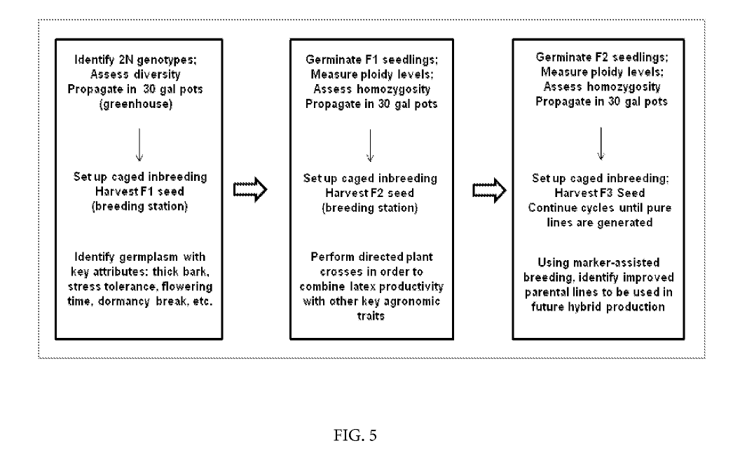

As described in FIG. 5, in a next phase of the breeding method of the invention, diploid guayule germplasm lines undergo further evaluation and improvement through selection and trait introgression, which is typically conducted under various environmental conditions. One of the primary objectives for this optional phase is to increase overall rubber yield and, in particular, increase the latex portion because this fraction can be used to produce hypoallergenic products. Other target traits of interest include improving rubber quality, seedling and mature plant vigor, time to maturity, bark thickness, dormancy break, plant architecture, regeneration following harvest by clipping (i.e. post-harvest regrowth), and tolerance to abiotic and/or biotic stresses such as, for example, salinity, drought, heat, cold, low availability of light and nutrient, diseases, insects and pests.

Such improvements are achieved by using conventional breeding techniques, marker-assisted breeding methods, as well as transgenesis.

As it will be appreciated by one skilled in the art, the breeding methodologies disclosed herein have far reaching ramifications not only for utility in the production of uniform hybrid seeds of guayule but for the development of similar plant materials in other facultative apomictic crops.

While a number of exemplary aspects and embodiments have been discussed above, those of skill in the art will recognize certain modifications, permutations, additions and sub-combinations thereof. It will be understood that elements of the embodiments described herein can be combined to make additional embodiments and various modifications may be made without departing from the spirit and scope of the invention. Accordingly, other embodiments, alternatives and equivalents are within the scope of the invention and claimed herein. Headings within the applications are solely for the convenience of the reader, and do not limit in any way the scope of the invention or its embodiments. It is therefore intended that the following appended claims and claims hereafter introduced are interpreted to include all such modifications, permutations, additions and sub-combinations as are within their true spirit and scope of the invention or its embodiments.

The discussion of the general methods given herein is intended for illustrative purposes only. Other alternative methods and embodiments will be apparent to those skilled in the art upon review of this disclosure. It should also be understood that the examples provided herein are offered to illustrate, but not limit, the invention.

What is claimed is:

1. A plant breeding method for the production of hybrid seed, said method comprising:
pollinating a female parent with pollen from a male parent to produce one or more F1 hybrid seeds on said female parent;
selecting an apomictic hybrid plant grown from said one or more F1 hybrid seeds;
clonally propagating said apomictic hybrid plant to produce a cloned apomictic plant line; and
growing one or more plants of said cloned apomictic plant line and collecting resulting apomictically-derived hybrid seeds from said grown plants,
wherein said female parent is a tetraploid plant or an essentially self-incompatible diploid plant, and said male parent is an tetraploid plant or a hexaploid plant.

2.-30. (canceled)

31. The method of claim 1, wherein said female parent is an essentially self-incompatible diploid parent and said male parent is a tetraploid male parent.

32. The method of claim 1, wherein said female parent is a tetraploid parent and said male parent is a hexaploid male parent.

33. The method of claim 1, wherein said one or more F1 hybrid seeds are triploid hybrid seeds, pentaploid hybrid seeds, or heptaploid hybrid seeds, or a combination thereof.

34. The method of claim 1, wherein said growing one or more plants of said apomictic plant line is performed in presence of at least one tetraploid plant.

35. The method of claim 1, wherein at least one of said female and male parents is pre-selected for high productivity.

36. The method of claim 1, wherein said apomictic plants grown from said one or more hybrid seeds are further selected for high productivity prior to said clonal propagation.

37. The method of claim 1, wherein at least one of said female and male parents are essentially homozygous plants or plants of inbred lines.

38. The method of claim 1, wherein said female and male parents are genetically distinct.

39. The method of claim 1, wherein at least one of said female and male parents are plants of a facultative apomictic species.

40. The method of claim 39, where said facultative apomictic plant species is a Parthenium argentatum species.

41. A hybrid seed produced by the method according to claim 1 or a hybrid plant grown from said hybrid seed.

42. The hybrid plant of claim 41, wherein said hybrid plant exhibits an improved target trait.

43. The hybrid plant of claim 41, further comprising a transgene.

44. A seed, a reproductive tissue, a vegetative tissue, a plant part, a biomass, or progeny of the hybrid plant according to claim 41.

45. A method for producing a plant-derived product, comprising obtaining a hybrid plant of claim 41, or a part thereof, and producing said plant-derived product therefrom.
